(19) United States
(12) Patent Application Publication
(10) Pub. No.: US 2003/0068339 A1
(43) Pub. Date: Apr. 10, 2003

(54) Veterinary florfenicol formulation that is syringeable under cold weather conditions

(75) Inventors: John R. Carpenter, Savannah, MO (US); Richard Mihalik, St. Joseph, MO (US)

Correspondence Address: Susan J. Wharton, SHOOK, HARDY & BACON L.L.P., 1200 Main Street, Kansas City, MO, 64105-2118, US

(73) Assignee: Phoenix Scientific, Inc.

(21) Appl. No.: 09/969,451

(22) Filed: Oct. 2, 2001

(51) Int. Cl. A61K 9/00; A61K 31/16; A61K 31/4015

(52) U.S. Cl. 424/400; 514/628; 514/424

(57) ABSTRACT

An antibiotic formulation for animals is provided. This formulation includes florfenicol, a preservative and N-methyl-2-pyrrolidone. The florfenicol and preservative are dissolved in the N-methyl-2-pyrrolidone solvent. The formulation is suitable for veterinary applications in colder temperatures. More specifically, it is usable during winter months because it has a lower cold temperature viscosity than previous formulations resulting in it having superior syringeability.

### STATEMENT REGARDING FEDERALLY SPONSORED RESEARCH OR DEVELOPMENT

Not Applicable.

### CROSS-REFERENCE TO RELATED APPLICATIONS

Not Applicable.

### BACKGROUND OF THE INVENTION

The present invention relates to an antibiotic formulation. More specifically, this formulation is able to be injected into animals at cold temperatures.

Bacterial infections can be a major problem for cattle and other livestock. Florfenicol is an antibacterial agent that is useful for treating, among other things, respiratory problems due to bacterial infections in livestock. When the weather is warmer, florfenicol formulations currently available may be used. However, such infections tend to occur during the winter months when temperatures are colder. One disadvantage of formulations currently available is that when temperatures become lower, a user is not able to easily withdraw florfenicol formulation from its vial and inject the florfenicol formulation into an animal. This is because the viscosity of the formulation becomes too high causing poor syringeability.

Because of the inferior syringeability of currently available florfenicol formulations, people have resorted to using alternative antibiotic solutions. The disadvantage with such solutions is that they may not be as effective in treating infections.

In order to overcome the disadvantages with formulations currently available, a formulation that includes florfenicol but is more easily injected by maintaining low viscosity, especially in cold weather, is needed. Still further, a florfenicol formulation that is able to be used all year long is needed.

### SUMMARY OF THE INVENTION

It is an object of the present invention to provide a low viscosity florfenicol formulation so that it can easily be loaded into and expelled from a syringe at cold temperatures.

It is a further object of the present invention to provide a method of making a florfenicol formulation with superior syringeability.

According to the present invention, the foregoing and other objects are achieved by an injectable antibiotic formulation that includes a mixture of florfenicol, a preservative, and a solvent. This formulation is made by mixing these components together until they have dissolved.

Additional objects, advantages and novel features of the invention will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### DETAILED DESCRIPTION OF THE PREFERRED EMBODIMENTS

The formulation of the present invention is an easily injectable florfenicol solution. This formulation includes, but is not limited to, an antibiotic, a preservative, and a solvent. More specifically, the formulation includes florfenicol as the antibiotic.

The preservative of the formulation of the present invention can be, but is not limited to, benzyl alcohol, ethyl alcohol, parabens such as methyl, ethyl and/or propylparaben, chlorobutanol, sodium benzoate, benzoic acid, myristyl-gamma-picolinium chloride, or combinations thereof. Preferably, benzyl alcohol is used as the preservative in the formulation.

The solvent used in the formulation of the present invention is a pyrrolidone solvent, namely, N-methyl-2-pyrrolidone (NM2P).

The antibiotic formulation of the present invention is made by mixing the florfenicol antibiotic and the preservative with the solvent to form a mixture. The antibiotic, preservative, and solvent are agitated together in a container until dissolved. Preferably, the preservative is agitated with the solvent before the antibiotic is introduced. Additional solvent may be added to the mixture after the components are dissolved. Preferably, the mixture is then filtered directly into bottles in order to render it sterile.

The formulation of the present invention includes about 5-60% weight per volume (w/v) florfenicol, about 5-100 mg preservative per ml of formulation (mg/ml preservative), and about 40-95% w/v solvent. Preferably, the formulation includes about 10-50% w/v florfenicol, about 10-50 mg/ml preservative, and about 50-90% w/v solvent. Most preferably, the formulation includes about 15-45% w/v florfenicol, about 10-20 mg/ml preservative, and about 55-85% w/v solvent.

The formulation of the present invention is of a much lower viscosity than florfenicol formulations currently available. Even at ambient temperatures, the viscosity of the preferred formulation of the present invention is nearly one third of the viscosity of florfenicol formulations currently available. At freezing temperatures, the preferred formulation of the present invention has a viscosity that is about 6 times lower than the viscosity of previous florfenicol formulations. These differences are shown in Table 1. Table 1 compares the viscosity of the formulation of the preferred embodiment of the present invention at room temperature and at freezing temperature to the viscosities of formulations currently on the market.

TABLE 1
Viscosities

| Temperature | Applicant's Formulation | Prior Art Formulation |
|---|---|---|
| 25° C. | 34 centipoises (cPs) | 95 cPs |
| 0° C. | 59 cPs | 388 cPs |

The viscosity of the formulation should be a maximum of about 60 cPs at 25° C. Preferably, it is less than about 50 cPs at 25° C., and most preferably, it is less than about 40 cPs at 25° C. The viscosity of the formulation should be a maximum of about 100 cPs at 0° C. Preferably, it is less than about 80 cPs at 0C, and most preferably, it is less than about 60 cPs at 0° C.

Applicant's formulation has better syringeability than formulations currently available. The improved syringeability of the formulation of the present invention allows it to be used at colder temperatures, such as during the winter. The florfenicol formulation of the present invention can even be used at temperatures below about 0° C. Preferably, the formulation is administered at temperatures between about −10 and 35° C. The syringeability of prior art formulations and the formulations of the preferred embodiment of the present invention are shown in Table 2. Both formulations included about 300 milligrams florfenicol per milliliter of formulation.

TABLE 2
Syringeabilities

| Needle | Temperature | Applicant's Formulation | Prior Art Formulation |
|---|---|---|---|
| 16 gauge | −20° C. | 5 mL expelled in 13 sec. | 5 mL expelled in 117 sec. |
| 16 gauge | −10° C. | 5 mL expelled in 3 sec. | 5 mL expelled in 200 sec. |
| 16 gauge | 0° C. | 5 mL expelled in 8 sec. | 5 mL expelled in 26 sec. |
| 16 gauge | 22.2° C. | 5 mL expelled in 3 sec. | 5 mL expelled in 10 sec. |
| 18 gauge | −20° C. | 5 mL expelled in 12 sec. | 5 mL expelled in 199 sec. |
| 18 gauge | −10° C. | 5 mL expelled in 4 sec. | 5 mL expelled in 125 sec. |
| 18 gauge | 0° C. | 5 mL expelled in 13 sec. | 5 mL expelled in 82 sec. |
| 18 gauge | 22.2° C. | 5 mL expelled in 4 sec. | 5 mL expelled in 22 sec. |
| 24 gauge | 0° C. | 5 mL expelled in 1 min., 19 | Approximately |
| | | sec. | 3/4 mL expelled in 5 min. |
| 24 gauge | 23° C. | 5 mL expelled in 42 sec. | 5 mL expelled in 7 min., 20 sec. |

Preferably, the formulation is parenterally administered with a syringe having about a ½ to 1 inch needle and having about a 16 to 24 gauge diameter. If such a needle is used, at least about 5 mL should be able to be expelled in about 2 minutes at 25° C., and at least about 5 mL should be able to be expelled in about 5 minutes at 0° C. Preferably, about 5 mL of the formulation can be expelled in less than about a minute at 25° C., and the same amount can be expelled in less than about two minutes at 0° C.

The florfenicol formulation of the present invention may be administered to animals including, but not limited to, bovine, equine, swine, wild ungulates, ovine, cows, horses, sheep, goats, poultry, donkeys, mules, zebras, cats and dogs. It may be used to fight infections caused by bacterial organisms. Preferably, it is administered by injection in a dosage of about 20 mg-40 mg per kilogram of animal and may be repeated in about 48 hours. Florfenicol in the formulation of the present invention is able to be injected intramuscularly or subcutaneously more easily than prior art formulations. This is because the formulation has a lower viscosity than previous formulations both at room temperature and at colder temperatures, as illustrated in Table 1. The most preferred formulation of the present invention is described in Example 1.

The following are examples of formulations that are within the scope of this invention. The formulation of Example 1 has been made. Examples 2 and 3 are hypothetical. These examples are not meant in any way to limit the scope of this invention.

### EXAMPLE 1

Florfenicol, the preservative benzyl alcohol, and the solvent NM2P were combined as follows to form the formulation of the present invention. First, a clean dry 16 liter high density polyethylene (HDPE) container was obtained and was calibrated to 10 liters. Next, approximately 7 kilograms of NM2P were added to the calibrated container. Once the NM2P was added, the solution was agitated with a mixer, adding approximately 200 grams of benzyl alcohol while the container was agitated. After adding the benzyl alcohol, approximately 3 kilograms of florfenicol were added to the solution during agitation until the florfenicol was dissolved. Thereafter, more NM2P was added until the formulation was 10 liters. After the components were added, the formulation was agitated for over 15 minutes. Once thoroughly mixed, the formulation was filtered directly into bottles under a laminar flow hood using sterile filtration techniques. Applicant used a Whatman® Polycap™ 75AS 0.2 micron nylon membrane filter with a glass micro fiber pre-filter.

### EXAMPLE 2

The formulation of the present invention is made as follows: Approximately 10 kilograms of NM2P are added to a container. Once the NM2P is added, it is agitated with a mixer. Next, approximately 300 grams of a mixture of methyl, ethyl and propylparabens are added while the container is being agitated. After adding the parabens, approximately 10 kilograms of florfenicol are added during agitation until the florfenicol is dissolved. Thereafter, more NM2P is added until the formulation is 10 liters. After the balance of solvent is added, the formulation is agitated for more than 15 minutes. Once thoroughly mixed, the formulation is filtered directly into bottles.

### EXAMPLE 3

The formulation of the present invention is made as follows: Approximately 15 kilograms of NM2P are added to a container. Once the NM2P is added, it is agitated with a mixer. Next, approximately 200 grams of ethyl alcohol are added while the container is being agitated. After adding the ethyl alcohol, approximately 1.5 kilograms of florfenicol are added during agitation until the florfenicol is dissolved. Thereafter, more NM2P is added until the formulation is 10 liters. After the balance of solvent is added, the formulation is agitated for at least 15 minutes. Once thoroughly mixed, the formulation is filtered directly into bottles.

From the foregoing, it will be seen that this invention is one well adapted to attain all the ends and objects hereinabove set forth together with other advantages which are obvious and inherent to the formulation. It will be understood that certain features and subcombinations are of utility and may be employed without reference to other features and subcombinations. This is contemplated by and is within the scope of the claims. Since many possible embodiments may be made of the invention without departing from the scope thereof, it is to be understood that all matter herein set forth is to be interpreted as illustrative and not in a limiting sense.

I claim:

1. An antibiotic formulation, comprising a mixture of:
florfenicol;
at least one preservative; and
N-methyl-2-pyrrolidone, wherein said florfenicol and said preservative are dissolved in said N-methyl-2-pyrrolidone to form an antibiotic formulation for veterinary use.

2. The formulation of claim 1, wherein said preservative is selected from the group consisting of benzyl alcohol, ethyl alcohol, methylparaben, ethylparaben, propylparaben, chlorobutanol, sodium benzoate, benzoic acid, myristyl-gamma-picolinium chloride, and combinations thereof.

3. The formulation of claim 1, wherein said formulation is comprised of about 5-60% w/v florfenicol, about 5-100 mg/ml preservative, and about 40-95% w/vN-methyl-2-pyrrolidone.

4. The formulation of claim 3, wherein said formulation is comprised of about 10-50% w/v florfenicol, about 10-50 mg/ml preservative, and about 50-90% w/vN-methyl-2-pyrrolidone.

5. The formulation of claim 4, wherein said formulation is comprised of about 15-45% w/v florfenicol, about 10-20 mg/ml preservative, and about 55-85% w/v N-methyl-2-pyrrolidone.

6. The formulation of claim 1, wherein the viscosity of said formulation is a maximum of about 100 cPs at 0° C.

7. The formulation of claim 1, wherein the viscosity of said formulation is a maximum of about 60 cPs at 25° C.

8. The formulation of claim 1, wherein at least about 5 ml can be expelled from a syringe in about 1 minute at 25° C.

9. The formulation of claim 1, wherein at least about 5 ml can be expelled from a syringe in about 2 minutes at 0° C.

10. A method of making an antibiotic formulation for administration through a syringe, comprising:
mixing florfenicol and a preservative with N-methyl-2-pyrrolidone to form a mixture.

11. The method of claim 10, wherein said mixing step further comprises:
agitating said florfenicol, preservative, and N-methyl-2-pyrrolidone together until dissolved.

12. The method of claim 11, wherein said preservative is agitated with said solvent before introducing said florfenicol.

13. The method of claim 12, wherein additional N-methyl-2-pyrrolidone is added after said florfenicol, preservative and N-methyl-2-pyrrolidone are mixed.

14. A method of administering an antibiotic formulation to an animal using a syringe, said method comprising:
putting a formulation comprised of florfenicol, at least one preservative, and N-methyl-2-pyrrolidone in said syringe; and
injecting said formulation into said animal parenterally using said syringe.

15. The method of claim 14, wherein said formulation is administered at a temperature between about −10 and 35° C.

16. The method of claim 15, wherein said formulation is administered at a temperature between about −10 and 15° C.

17. The method of claim 14, wherein said formulation is administered to animals selected from the group consisting of bovine, equine, swine, wild ungulates, ovine, cows, horses, sheep, goats, poultry, donkeys, mules, zebras, cats and dogs.

18. The method of claim 17, wherein said formulation is administered intramuscularly or subcutaneously in a dosage of about 20-40 mg per kilogram of animal.

19. The method of claim 18, wherein said administration is repeated in about 48 hours.
